(19) United States
(12) Patent Application Publication
(10) Pub. No.: US 2024/0032848 A1
(43) Pub. Date: Feb. 1, 2024

(54) APPARATUS AND METHOD FOR DETECTING MOTOR HOTSPOT POSITION BY USING BRAINWAVE, AND TRANSCRANIAL ELECTRICAL STIMULATION APPARATUS USING SAME

(71) Applicant: Korea University Research and Business Foundation, Sejong Campus, Sejong (KR)

(72) Inventors: Han-Jeong HWANG, Sejong-si (KR); Ga-Young CHOI, Sejong-si (KR); Won-seok KIM, Seongnam-si (KR); Nam-Jong PAIK, Seongnam-si (KR)

(73) Assignee: Korea University Research and Business Foundation, Sejong Campus, Sejong (KR)

(21) Appl. No.: 18/014,007

(22) PCT Filed: Feb. 19, 2021
(86) PCT No.: PCT/KR2021/002118

§ 371 Date: Dec. 30, 2022

(30) Foreign Application Priority Data
Jun. 30, 2020 (KR) 10-2020-0080383
Oct. 16, 2020 (KR) 10-2020-0133831

(51) Int. Cl. A61B 5/369; A61B 5/00; A61N 2/00; A61N 1/04

(52) U.S. Cl. CPC A61B 5/369; A61B 5/7264; A61N 2/006; A61B 5/6803; A61N 1/0456

(57) ABSTRACT

Disclosed are a motor hotspot location detecting device and a motor hotspot location detecting method using brainwaves, by which a location of a motor hotspot may be detected based on brainwaves, and a transcranial electrical stimulation device using the same. The motor hotspot location detecting device using brainwaves includes a plurality of brainwave measuring electrode that measures brainwaves generated in the target object at different locations and collects brainwave data, and a motor hotspot location detecting part that detects the location of the motor hotspot based on the brainwave data collected by the plurality of brainwave measuring electrodes.

### TECHNICAL FIELD

The inventive concept relates to a motor hotspot location detecting device and a motor hotspot location detecting method, and more particularly, to a motor hotspot location detecting device and a motor hotspot location detecting method using brainwaves, by which a location of a motor hotspot may be detected based on brainwaves, and a transcranial electrical stimulation device using the same.

The inventive concept is a study result of “Development of a BCI based Brain Recognition Computing Technology of Recognizing Intention of Person by Using Deep Learning (study No. 2017-0-00451)” performed with a support of finances of Korean Government by Information and Communications Planning and Evaluation Institute.

Furthermore, the inventive concept is a study result of a basic research business (2020R1A4A1017775) performed with a support of finances of Ministry of Science and ICT by National Research Foundation of Korea.

### BACKGROUND ART

The transcranial electrical stimulation (tES) is a technology of non-invasively applying a current, such as a fine direct current or alternating current, to a scalp of a subject person, such as an exercise function rehabilitation patient, and it is known to have an effect of improving an exercise function disorder that often appears after various brain injuries, such as strokes, occur or alleviating nervous pains or a mental disorder such as depression.

A location, to which a transcranial electrical stimulation is applied, is different according to the diseases of a subject person and a treatment purpose. The transcranial electrical stimulation is used together with a rehabilitation exercise to maximize an exercise rehabilitation effect, and generally, a stimulus is applied to a hotspot of the brain. Conventionally, to search for hotspots for individuals, a scheme of searching for a transcranial magnetic stimulation (TMS) application location (a brain/scalp area portion), such as a specific body portion such as a finger, at which a motor evoked potential (MEP) occurs most explicitly and applying a brain electrical stimulus is applied to the corresponding location. That is, an electrical stimulation electrode is attached to a location of a motor hotspot on a scalp, which is found through a transcranial magnetic stimulation scheme to apply a current for a transcranial electrical stimulation.

According to a conventional method for selecting a location of a motor hotspot for a transcranial electrical stimulation, a patient has a troublesomeness of having to go to a hospital provided with transcranial magnetic stimulation equipment all the time because the transcranial magnetic stimulation equipment is high-priced and cannot be carried by the patient. In particular, most of the patients who require a transcranial electrical stimulation treatment have a difficulty in moving their bodies, the inconveniences of the patients are much severer and burdens on costs are high. Furthermore, because experiential determinations of experts, such as doctors, have to be accompanied to determine the location of the motor hotspot based on a motor evoked potential (MEP) that is induced in the transcranial magnetic stimulation scheme, the location of the motor hotspot may be differently determined according to the skillfulness of the experts.

### DETAILED DESCRIPTION OF THE INVENTION

### Techinical Problem

The inventive concept provides a motor hotspot location detecting device and a motor hotspot location detecting method using brainwaves, by which a location of a motor hotspot may be detected based on brainwaves, a transcranial electrical stimulation device using the same, and a recording medium, in which a program for detecting a location of a motor hotspot by using brainwaves is recorded.

The inventive concept also provides a motor hotspot location detecting device and a motor hotspot location detecting method using brainwaves, by which a brain electrical stimulation treatment may be performed without any temporal/spatial restriction while not being free of expertise of a user by automatically detecting a location of a motor hotspot for an individual through measurement of brainwaves instead of a bothersome transcranial magnetic stimulation (TMS) and providing a location of a transcranial electrical stimulation, and an exercise function rehabilitation and brain function improving effect may be enhanced through home cares of brain electrical stimulation rehabilitation and consistent treatments, a transcranial electrical stimulation device using the same, and a recording medium, in which a program for detecting a location of a motor hotspot by using brainwaves is recorded.

The inventive concept also provides a motor hotspot location detecting device and a motor hotspot location detecting method using brainwaves, by which a target location for a transcranial electrical stimulation may be automatically determined by any one easily and precisely by using a convolutional neural network based on brainwave data collected by a user without any database of a high capacity, a transcranial electrical stimulation device using the same, and a recording medium, in which a program for detecting a location of a motor hotspot by using brainwaves is recorded.

### Technical Solution

A motor hotspot location detecting device using brainwaves for detecting a location of a motor hotspot that is a target location for a transcranial electrical stimulation of a scalp of a target object according to an embodiment of the inventive concept includes a plurality of brainwave measuring electrode that measures brainwaves generated in the target object at different locations and collect brainwave data, and a motor hotspot location detecting part that detects the location of the motor hotspot based on the brainwave data collected by the plurality of brainwave measuring electrodes.

A transcranial electrical stimulation device for detecting a location of a motor hotspot of a target object and applying a transcranial electrical stimulation to a scalp portion corresponding to the location of the motor hotspot according to an embodiment of the inventive concept includes a head mounted body provided in a form that is mountable on the scalp of the target object, a plurality of brainwave measuring electrodes that measures brainwaves generated in the target object at different locations and collects brainwave data and distributed and disposed on an inner surface of the head mounted body, a motor hotspot location detecting part that detects the location of the motor hotspot based on the brainwave data collected by the plurality of brainwave measuring electrodes, and a transcranial electrical stimulation electrode provided in the head mounted body such that the transcranial electrical stimulation is applied onto the scalp of the target object.

A motor hotspot location detecting method using brainwaves for detecting a location of a motor hotspot that is a target location for a transcranial electrical stimulation of a scalp of a target object according to an embodiment of the inventive concept includes collecting brainwave data obtained by measuring brainwaves generated in the target object at different locations, and detecting the location of the motor hotspot based on the brainwave data, by a motor hotspot location detecting part.

According to an embodiment of the inventive concept, a non-transitory computer readable recording medium, in which a program for executing the motor hotspot location measuring method using brainwaves is recorded, is provided.

### Advantageous Effects of the Invention

According to the embodiment of the inventive concept, a motor hotspot location detecting device and a motor hotspot location detecting method using brainwaves, by which a location of a motor hotspot may be detected based on brainwaves, a transcranial electrical stimulation device using the same, and a recording medium, in which a program for detecting a location of a motor hotspot by using brainwaves is recorded, are provided.

Furthermore, according to the embodiment of the inventive concept, a brain electrical stimulation treatment may be performed without any temporal/spatial restriction while not being free of expertise of a user by automatically detecting a location of a motor hotspot for an individual through measurement of brainwaves instead of a bothersome transcranial magnetic stimulation (TMS) and providing a location of a transcranial electrical stimulation, and an exercise function rehabilitation and brain function improving effect may be enhanced through home cares of brain electrical stimulation rehabilitation and consistent treatments.

Furthermore, according to the embodiment of the inventive concept, a target location for a transcranial electrical stimulation may be automatically determined by any one easily and precisely by using a convolutional neural network based on brainwave data collected by a user without any database of a high capacity.

### DESCRIPTION OF THE DRAWINGS

FIG. 1 is a perspective view illustrating an in-use state of a transcranial electrical stimulation device including a motor hotspot location detecting device using brainwaves according to an embodiment of the inventive concept.

FIG. 2 is a plan view of a motor hotspot location detecting device using brainwaves according to an embodiment of the inventive concept.

FIG. 3 is a diagram of a motor hotspot location detecting device using brainwaves according to an embodiment of the inventive concept.

FIG. 4 is a diagram of a motor hotpot location detecting part that constitutes a motor hotspot location detecting device using brainwaves according to an embodiment of the inventive concept.

FIG. 5 is an exemplary view of brainwave data collected by a motor hotspot location detecting device using brainwaves according to an embodiment of the inventive concept.

FIG. 6 is a diagram of a feature data calculating part that constitutes a motor hotspot location detecting device using brainwaves according to an embodiment of the inventive concept.

FIG. 7 is an exemplary view of power spectrum densities generated by a motor hotspot location detecting device using brainwaves according to an embodiment of the inventive concept.

FIG. 8 is a graph depicting hotspot location errors calculated based on power spectrum densities corresponding to six frequency bands of a feature data calculating part.

FIG. 9 is a conceptual view of an artificial neural network that constitutes a motor hotspot location detecting device using brainwaves according to an embodiment of the inventive concept.

FIG. 10 is a conceptual view illustrating a process of learning an artificial neural network that constitutes a motor hotspot location detecting device using brainwaves according to an embodiment of the inventive concept.

FIGS. 11A and 11B are exemplary views illustrating locations of a plurality of brainwave measuring electrodes that constitute a motor hotspot location detecting device using brainwaves according to an embodiment of the inventive concept, and a location of a motor hotspot calculated by a transcranial magnetic stimulation (TMS).

FIGS. 12A to 12D are exemplary views illustrating arrangement of a plurality of brainwave measuring electrodes that constitute a motor hotspot location detecting device according to various embodiments of the inventive concept.

FIG. 13 is an exemplary view of a transcranial electrical stimulation electrode that constitutes a transcranial electrical stimulation device according to an embodiment of the inventive concept.

FIG. 14 is an exemplary view of a transcranial electrical stimulation electrode that constitutes a transcranial electrical stimulation device according to another embodiment of the inventive concept.

FIG. 15 is a flowchart of a motor hotspot location detecting method using brainwaves according to an embodiment of the inventive concept.

FIG. 16 is a view obtained by comparing a location of a motor hotspot detected through a motor hotspot location detecting method using brainwaves according to an embodiment of the inventive concept and a location of a motor hotspot detected through a transcranial magnetic stimulation scheme.

FIG. 17 is a graph depicting a transcranial electrical stimulation error calculated based on power spectrum densities after extracting the power spectrum densities corresponding to six frequency bands as features.

FIG. 18 is a graph depicting an average transcranial electrical stimulation location detection error for pre-processing operations.

FIG. 19 is a conceptual view of a convolutional neural network that constitutes a brainwave based transcranial electrical stimulation location determining device using a convolutional neural network according to an embodiment of the inventive concept.

FIG. 20 is a graph depicting a transcranial electrical stimulation location error calculated according to an embodiment of the inventive concept while the number of channels applied to an analysis is gradually decreased from a total of sixty three channels to nine channels with respect to a motion area.

### BEST MODE

The above and other aspects, features, and advantages of the inventive concept will become apparent from the following description of the following embodiments given in conjunction with the accompanying drawings. However, the inventive concept is not limited by the embodiments disclosed herein but will be realized in various different forms, and the embodiments are provided only to make the disclosure of the inventive concept complete and fully inform the scope of the inventive concept to an ordinary person in the art, to which the inventive concept pertains, and the inventive concept will be defined by the scope of the claims. The same reference numerals denote the same elements throughout the specification.

Throughout the specification, when it is described that a part includes a component, it may mean that the part may further include second component without excluding the second component unless a specially contradictory description is made. The term ‘unit’ or ‘module’ used herein is a unit for processing at least one function or operation, and for example, may refer to software, an FPGA, or a hardware component. The functions provided by the ‘unit’ or the ‘module’ may be separately performed by a plurality of components or may be integrated with another additional component. The ‘unit’, ‘-er(or)’, and “module” herein are not necessarily limited to software or hardware, and may be constituted in a storage medium that may perform addressing, and may be configured to reproduce one or more processors. Hereinafter, embodiments of the inventive concept will be described in detail with reference to the drawings.

A motor hotspot location detecting device and a motor hotspot location detecting method using brainwaves according to an embodiment of the inventive concept are adapted to automatically determine a location of a motor hotspot that is a transcranial electrical stimulation target location for a target object.

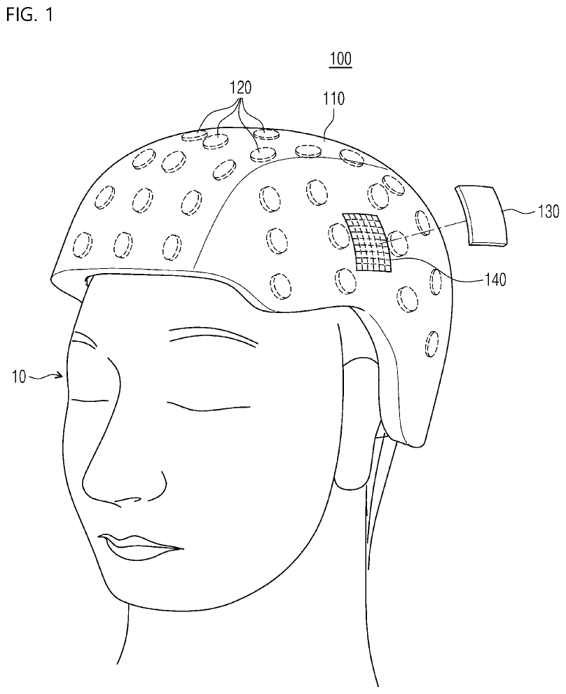

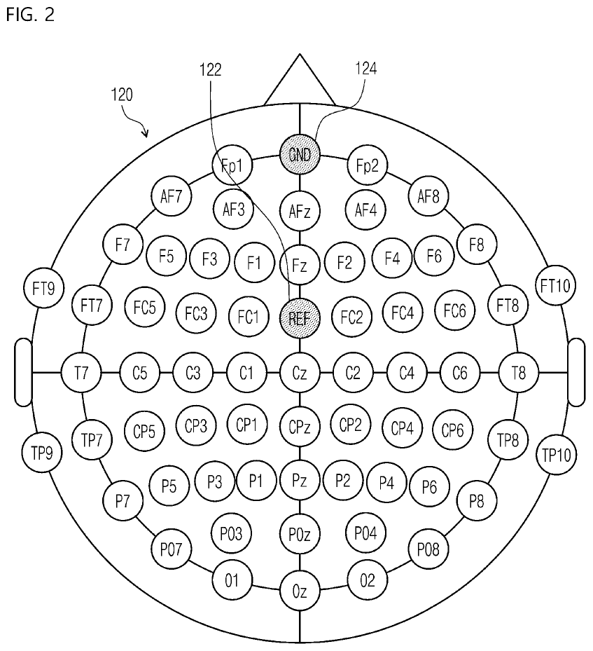

FIG. 1 is a perspective view illustrating an in-use state of a transcranial electrical stimulation device including a motor hotspot location detecting device using brainwaves according to an embodiment of the inventive concept. FIG. 2 is a plan view of the motor hotspot location detecting device using brainwaves according to an embodiment of the inventive concept.

The transcranial electrical stimulation device according to an embodiment of the inventive concept is described as an embodiment of the motor hotspot location detecting device using brainwaves, and it is described in advance that the motor hotspot location detecting device and the motor hotspot location detecting method using brainwaves according to an embodiment is not limited to application to the transcranial electrical stimulation device.

Referring to FIGS. 1 and 2, the transcranial electrical stimulation device according to an embodiment of the inventive concept includes a motor hotspot location detecting device 100. The motor hotspot location detecting device 100 is configured to detect a location of a motor hotspot that is a target location for a transcranial electrical stimulation (tES) for a scalp of a target object 10.

The motor hotspot location detecting device 100 may include a head mounted body 110, a plurality of brainwave measuring electrodes 120, a motor hotspot location detecting part 130, and a transcranial electrical stimulation electrode 140.

The head mounted body 110 may be provided in a form that may be mountable on the scalp of the target object 10. The head mounted body 110 may be mounted on at least a portion of the scalp of the target object 10 in a form, such as a headset or a cap.

The target object 10, for example, may be a head including a brain of an exercise function rehabilitation patient who undergoes an exercise function disorder that appears after various brain injuries, such as a stroke, occur, but the inventive concept is not limited thereto.

The plurality of brainwave measuring electrodes 120 are configured to measure brainwaves generated by the target object 10 when a subject person performs a specific motion (for example, a rehabilitation related action) and collect brainwaves. The plurality of brainwave measuring electrodes 120 may be distributed and disposed on an inner surface of the head mounted body 110 such that the brainwave data are collected at different locations on the scalp of the target object 10.

In addition to the plurality of brainwave measuring electrodes 120, a reference electrode 122 and a ground electrode 124 may be provided in the head mounted body 110.

The reference electrode 122 may be an electrode for providing a potential value that is a reference for brainwaves. That is, the potential value of the brainwave measuring electrode 120 with reference to the potential value of the reference electrode 122 may be acquired as brainwave data.

In other words, a potential difference between a reference signal that is a potential value of the reference electrode 122 and measurement signals for locations of the brainwave measuring electrodes 120 may be finally collected as the brainwave data for the channels. However, when an average value for the brainwave measuring electrodes 120 is used as the reference value, a separate reference electrode may not be attached.

The brainwave data may be brainwave signals that are collected by the brainwave measuring electrode 120, and may be brainwave signals that are collected by the brainwave measuring electrode 120 with reference to the brainwave signals collected by the reference electrode 122.

The ground electrode 124 may be an electrode that is attached for the purpose of adjusting a potential difference of the equipment itself to 0 point to provide a precise potential difference between the brainwave measuring electrodes 120 and the reference electrode 122.

The motor hotspot location detecting part 130 is configured to detect a location of a motor hotspot of the target object 10 based on the brainwave data collected by the plurality of brainwave measuring electrodes 120.

The motor hotspot location detecting part 130 may include at least one processor, and a memory that stores a motor hotspot location detection algorithm. The motor hotspot location detecting part 130 may be provided in the head mounted body 110, and may be connected to the brainwave measuring electrodes 120 and a transcranial electrical simulation electrode 140 via electrical signals.

In an embodiment, after an artificial neural network model is constructed, the motor hotspot location detecting part 130 may detect the location of the motor hotspot based on only the brainwave data collected for the channels by the plurality of brainwave measuring electrodes 120, except for 3-dimensional location information on the motor hotspot measured through a transcranial magnetic simulation scheme.

In an embodiment, the motor hotspot location detecting part 130 may be disposed in a one-module form on an outside of the transcranial electrical simulation electrode 140 to increase spatial utility, but a disposition location of the motor hotspot location detecting part 130 is not limited to the illustration.

The transcranial electrical stimulation electrode 140 may be provided in the head mounted body 110 such that a transcranial electrical stimulation is applied on to the scalp of the target object 10. The transcranial electrical stimulation electrode 140 may apply a transcranial electrical stimulus to a scalp corresponding to a motor hotspot location of the target object 10, which is detected by the motor hotspot location detecting part 130.

The transcranial electrical stimulation electrode 140 may non-invasively apply a current (a transcranial electrical stimulation), such as a fine direct current or alternating current, to a motion hotspot portion that is a brain exercise area of subject person (for example, an exercise rehabilitation patient). Accordingly, an exercise rehabilitation effect of a subject person, such as an exercise rehabilitation patient, may be enhanced.

In an embodiment, the transcranial electrical stimulation electrode 140 may include an electrode set including two electrodes of a positive electrode and a negative electrode. An electrical stimulation location by the positive electrode and the negative electrode may be determined according to the location of the motor hotspot.

For example, any one of the positive electrode and the negative electrode may apply an electrical stimulation at the location of the motor hotspot, and the other one may apply an electrical signal to a location that is spaced apart from the location of the motor hotspot by a preset distance according to the location of the motor hotspot.

The transcranial electrical stimulation electrode 140 may be configured to apply an electrical stimulus through any one of a plurality of arranged electrodes, and move the electrode to the location of the motor hotspot to apply an electrical stimulation according to the location of the motor hotspot.

According to the above-described embodiment of the inventive concept, the brainwave data may be collected by the plurality of brainwave measuring electrodes 120 provided in the head mounted body 110 mounted to the subject person by himself or herself, the precise location of the motor hotspot may be detected, and the transcranial electrical stimulation may be applied.

Accordingly, the subject person may obtain an improved exercise rehabilitation effect by applying a brain electrical stimulus to a precise brain exercise area for an individual even though he or she does not visit a hospital to find a brain domain, in which a motor evoked potential (MEP) is caused most explicitly due to the transcranial magnetic stimulation (TMS), and inconveniences due to visit to the hospital may be reduced and expenditure of medical costs may be reduced.

Furthermore, according to an embodiment of the inventive concept, through simple measurement of brainwaves instead of a bothersome transcranial magnetic stimulation measuring scheme after the artificial neural network is constructed, a brain electrical stimulation treatment may be performed at the location of the motor hotspot without being restricted by an expertise of a user and without any temporal or spatial limitation by automatically detecting the location of the motor hotspot for an individual and providing the transcranial electrical stimulation, and an exercise function rehabilitation and brain function improving effect may be enhanced through home care of the brain electrical stimulation rehabilitation and consistent treatments.

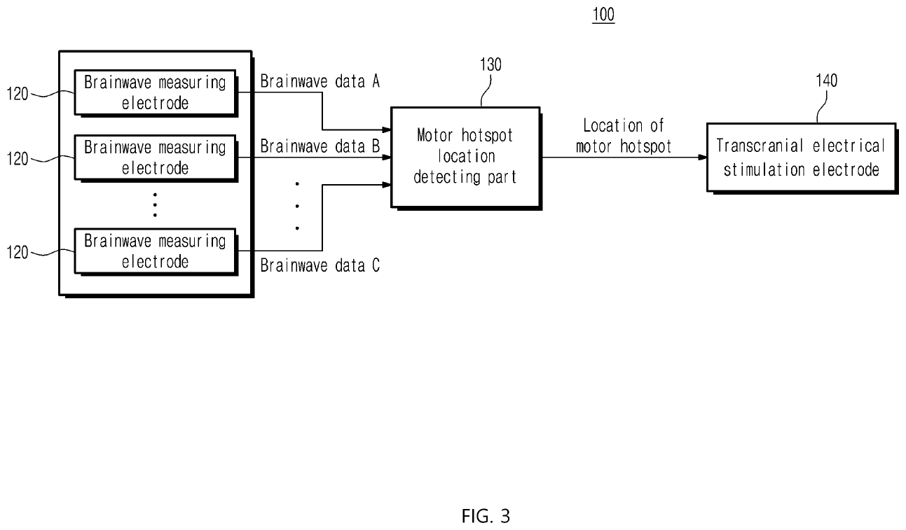

FIG. 3 is a diagram of the motor hotspot location detecting device using brainwaves according to an embodiment of the inventive concept. Referring to FIGS. 1 and 3, the motor hotspot location detecting device 100 may include the plurality of brainwave measuring electrodes 120 and the motor hotspot location detecting part 130.

The motor hotspot location detecting part 130 may include an artificial neural network that calculates the location of the motor hotspot based on brainwave data of time domains collected for the channels allocated to the plurality of brainwave measuring electrodes 120 or feature data extracted from the brainwave data.

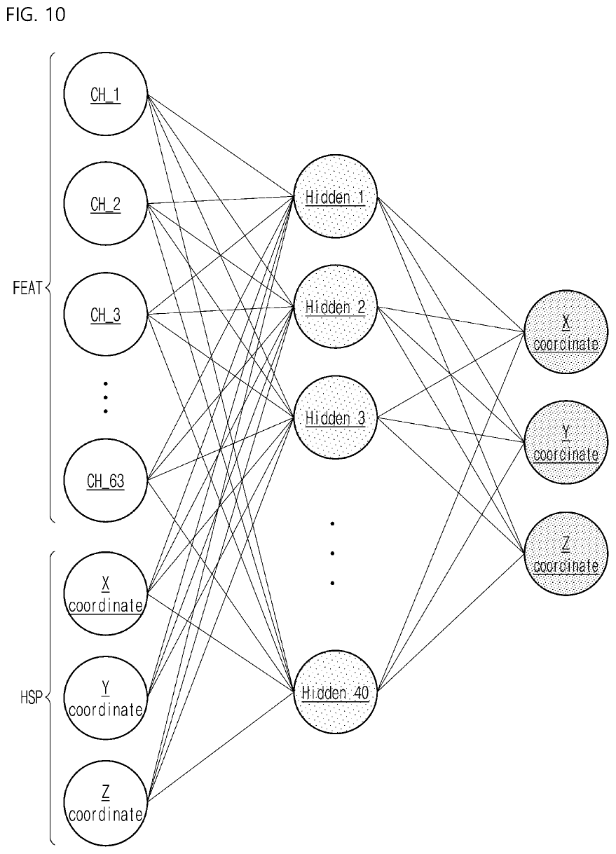

An artificial neural network model of the motor hotspot location detecting part 130 may perform learning based on the location of the motor hotspot corresponding to the brainwave data collected for the plurality of channels by the plurality of brainwave measuring electrodes 120 and the transcranial magnetic stimulation data measured by the transcranial magnetic stimulation. An artificial neural network leaning method of the motor hotspot location detecting part 130 will be described later with reference to FIG. 10.

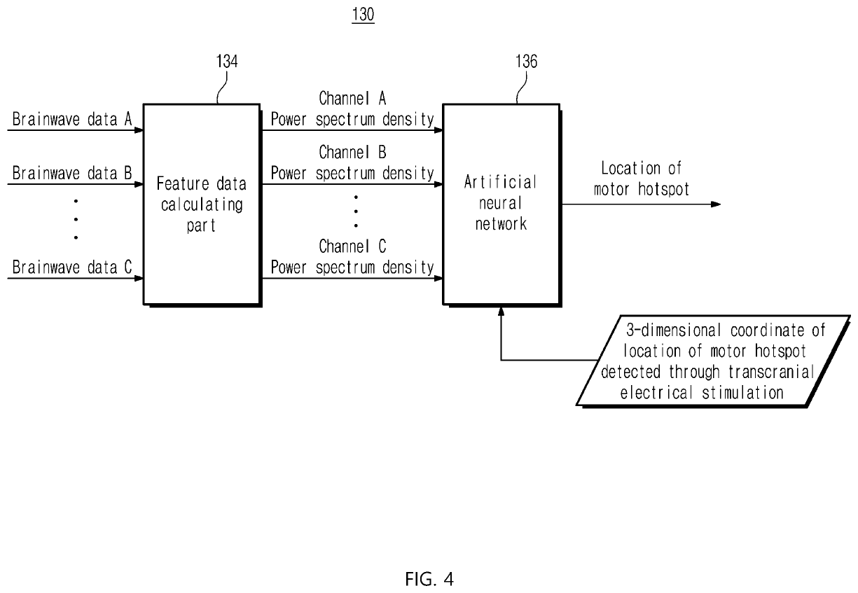

FIG. 4 is a diagram of the motor hotpot location detecting part that constitutes the motor hotspot location detecting device using brainwaves according to an embodiment of the inventive concept. Referring to FIGS. 1 to 4, the motor hotspot location detecting part 130 may include a feature data calculating part 134 and an artificial neural network 136.

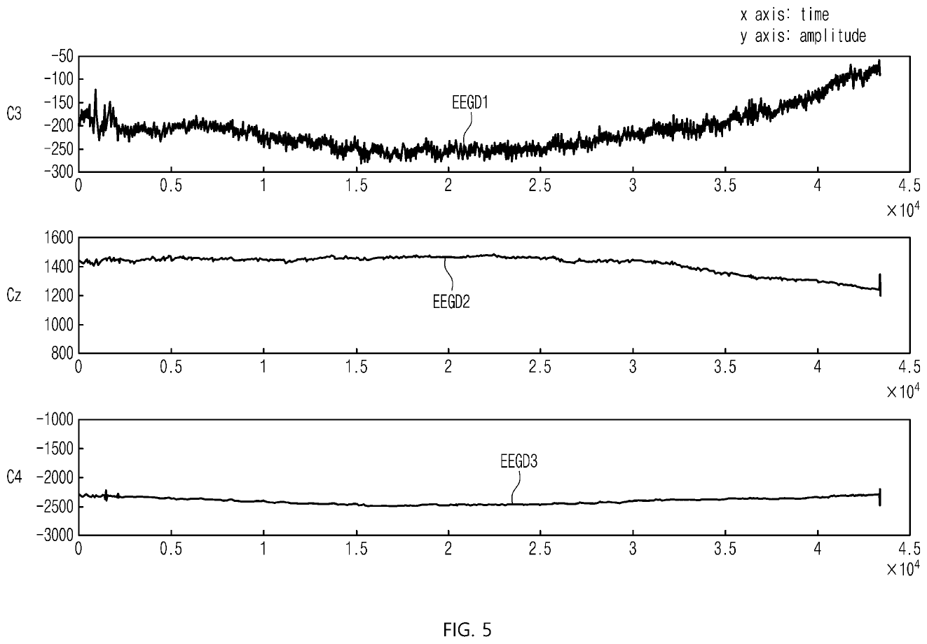

FIG. 5 is an exemplary view of brainwave data collected by the motor hotspot location detecting device using brainwaves according to an embodiment of the inventive concept. The three brainwave data illustrated in FIG. 5 are brainwave data collected by, among the plurality of brainwave measuring electrodes illustrated in FIG. 2, electrode ‘c3’, electrode ‘cz’, and electrode ‘c4’.

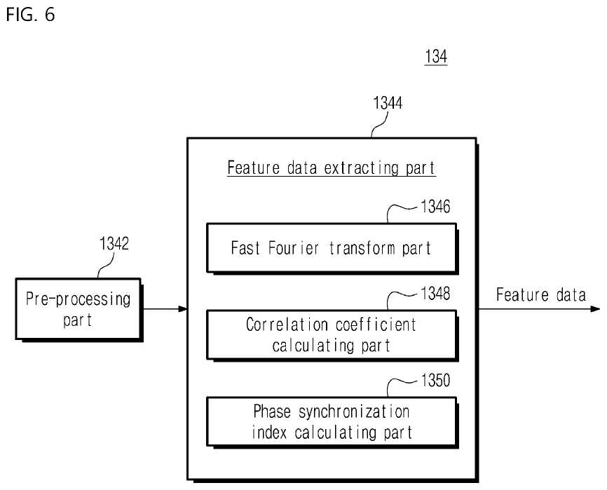

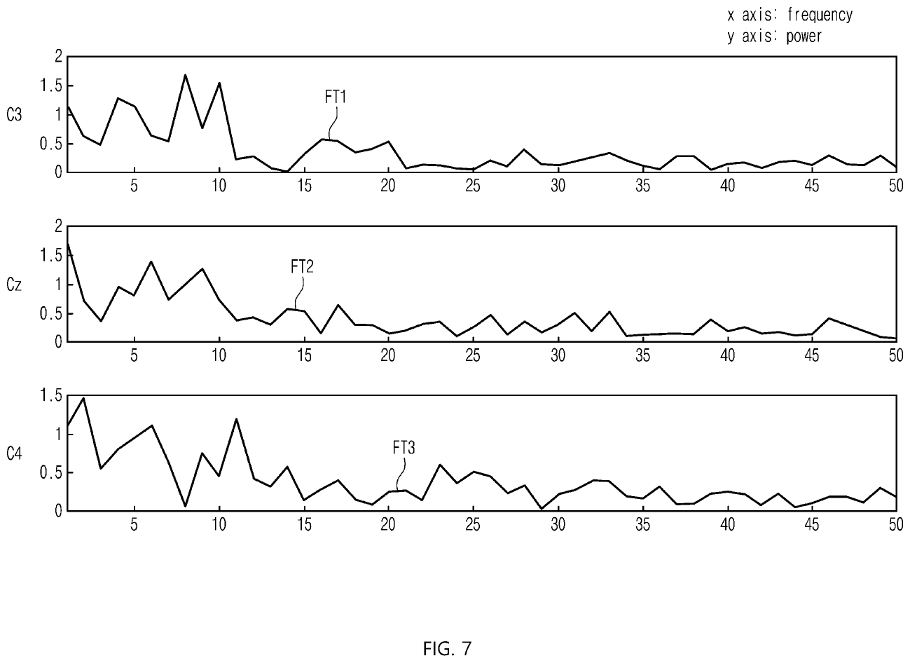

FIG. 6 is a diagram of the feature data calculating part that constitutes the motor hotspot location detecting device using brainwaves according to an embodiment of the inventive concept. FIG. 7 is an exemplary view of power spectrum densities (PSDs) generated by the motor hotspot location detecting device using brainwaves according to an embodiment of the inventive concept.

Referring to FIGS. 3 to 7, in an embodiment, the feature data calculating part 134 may convert brainwave data EEGD1, EEGD2, and EEGD3 of the time domains collected for the plurality of channels allocated to the plurality of brainwave measuring electrodes 120 into frequency area data and may generate power spectrum densities FT1, FT2, and FT3 for the channels.

In an embodiment of the inventive concept, the feature data calculating part 134 may convert the brainwave data EEGD1, EEGD2, and EEGD3 to the power spectrum densities FT1, FT2, and FT3 through a fast Fourier transform (FFT). The feature data calculating part 134, for example, may convert the brainwave data into the power spectrum densities according to Equation 1 as follows.

f(ϵ)=∫_{-∞}^{∞}f(x)e^{-2πixϵ}dlx,ϵ,frequency) [Equation 1]

In Equation 1, f(x) denotes the brainwave data of the equally divided time domains, f(ε) denotes a power spectrum density of a frequency domain obtained by converting the brainwave data of the time domain, “x” denotes time, and ε denotes a frequency.

In an embodiment, the feature data calculating part 134 may convert the brainwave data for the plurality of channels into the power spectrum densities FT1, FT2, and FT3 to calculate feature data including the same.

In another embodiment of the inventive concept, the feature data calculating part 134 may convert the brainwave data of the time domains into the power spectrum densities by using a wavelet transform according to Equation 2 as follows and an autoregressive scheme according to Equation 3 as follows, in addition to the fast Fourier transform.

$$X ( a , b ) = \frac{1}{\sqrt{a}} {\int }_{- ∞}^{∞} \overline{Ψ ( \frac{t - b}{a} )} x ( t ) dt , ( a , scaling ; b . time ) \qquad [ Equation 2 ]$$

In Equation 2, X(a,b) denotes data obtained by wavelet-converting the brainwave data of the equally divided time domains, x(t) denotes the brainwave data of the equally divided time domains, Ψ is a wavelet transform function, and (a,b) denotes (a scale factor, a time factor).

$$S ( f ) = \frac{{σ}_{z}^{2}}{{\vert 1 - {\sum }_{k = 1}^{p} {φ}_{k} {e}^{- i 2 π fk} \vert}^{2}} \qquad [ Equation 3 ]$$

In Equation 3, S(f) denotes data of the frequency domains obtained by converting the brainwave data of the equally divided time domains through an autoregressive scheme, “f” denotes a frequency, “φ” denotes an autoregressive function, σ²z is a variance of the brainwave data, “p” denotes the number of the channels of the brainwave data, and “k” denotes an integer (1≤k≤p).

The feature data for the channels calculated by the feature data calculating part 134 may be input to input nodes of the artificial neural network 136 to calculate the location of the motor hotspot. The artificial neural network 136 may calculate the location of the motor hotspot based on the feature data including the power spectrum densities calculated for the plurality of channels by the feature data calculating part 134.

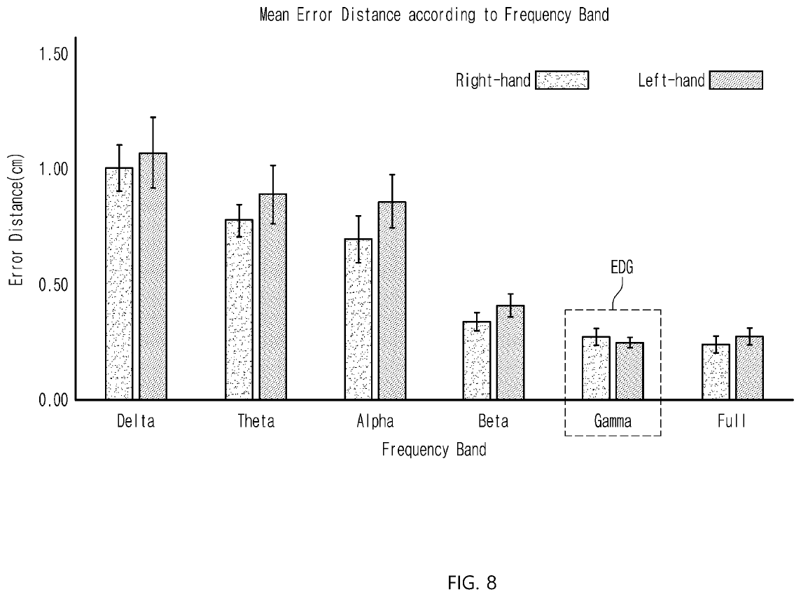

FIG. 8 is a graph depicting hotspot location errors calculated based on power spectrum densities corresponding to six frequency bands of the feature data calculating part. Referring to FIGS. 3, 4, 7, and 8, the feature data calculating part 134 may include a pre-processing part 1342 and a feature data extracting part 1344.

The pre-processing part 1342 may perform pre-processing, such as removal of noise, on the brainwave data for the channels collected by the plurality of brainwave measuring electrodes 120.

The feature data extracting part 1344 may extract the feature data including the power spectrum densities, a correlation between the brainwave data, and/or the phase synchronization index, from the brainwave data for the channels, to which the pre-processing, such as removal of noise, has been performed by the pre-processing part 1342.

In an embodiment of the inventive concept, the feature data extracting part 1344 may include a fast Fourier transform part 1346, a correlation coefficient calculating part 1348, and/or a phase synchronization index calculating part 1350.

The fast Fourier transform part 1346 may generate the power spectrum densities through fast Fourier transform of the brainwave data for the channels and may extract the feature data including the same. The feature data including the power spectrum densities extracted by the feature data extracting part 1344 may be input to the artificial neural network to detect the location of the motor hotspot.

The correlation coefficient calculating part 1348 may calculate the correlation coefficient that represents a correlation between the plurality of brainwave data. The feature data including the correlation coefficient extracted by the feature data extracting part 1344 may be input to the artificial neural network to detect the location of the motor hotspot.

The phase synchronization index calculating part 1350 may calculate a phase synchronization index between the plurality of brainwave data. The feature data including the phase synchronization index extracted by the feature data extracting part 1344 may be input to the artificial neural network to detect the location of the motor hotspot.

In the graphs of FIG. 8, delta denotes an error of the location of the motor hotspot calculated based on power spectrum densities in a frequency range of 1 to 4 Hz after the power spectrum densities are extracted, theta denotes an error of the location of the motor hotspot calculated based on power spectrum densities in a frequency range of 4 to 8 Hz after the power spectrum densities are extracted, and alpha denotes an error of the location of the motor hotspot calculated based on power spectrum densities in a frequency range of 8 to 13 Hz after the power spectrum densities are extracted.

Furthermore, beta denotes an error of the location of the motor hotspot calculated based on power spectrum densities in a frequency range of 13 to 30 Hz after the power spectrum densities are extracted, gamma denotes an error of the location of the motor hotspot calculated based on power spectrum densities in a frequency range of 30 to 50 Hz after the power spectrum densities are extracted, and full denotes an error of the location of the motor hotspot calculated based on power spectrum densities in a frequency range of 1 to 50 Hz after the power spectrum densities are extracted.

Then, the errors of the motor hotspot location for the frequency bands are obtained through a method of extracting the power spectrum densities for the frequency bands through a process of performing fast Fourier transform (FFT) on the brainwave data collected for the channels by the brainwave measuring electrodes, and comparing the location of the motor hotspot obtained by inputting the extracted power spectrum densities for the channels to the artificial neural network and a location of an exercised brain domain, which is found through the transcranial magnetic stimulation (TMS).

It may be seen from the result illustrated in FIG. 8 that the measurement error (EDG) of the location of the motor hotspot is lowest when a frequency range of 1 to 50 Hz is used. Furthermore, it may be seen that when the location of the motor hotspot is calculated by extracting the power spectrum density of the frequency band (a frequency range of 30 to 50 Hz) corresponding to gamma is calculated, the measurement error (EDG) of the location of the motor hotspot is small as compared with when the power spectrum density of another frequency band is extracted, and the measurement error (EDG) of the location of the motor hotspot is almost the same as compared with when the power spectrum density of a frequency range of 1 to 50 Hz is used.

The frequency range of the power spectrum density for extracting the feature data may be freely set to various frequency bands without being limited to the above-mentioned frequency band.

Furthermore, the location of the motor hotspot may be detected by extracting other feature data, such as the correlation between brainwaves or the phase synchronization index, which has been mentioned above, in addition to the power spectrum densities. Furthermore, the power spectrum densities may be extracted through a wavelet transform or an autoregressive scheme, in addition to a fast Fourier transform.

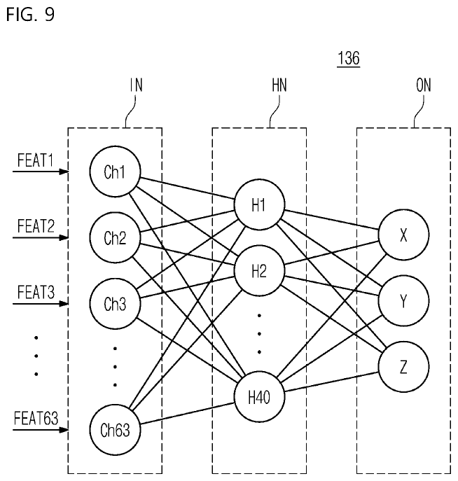

FIG. 9 is a conceptual view of the artificial neural network that constitutes the motor hotspot location detecting device using brainwaves according to an embodiment of the inventive concept. Referring to FIGS. 4 and 9, the artificial neural network 136 may calculate the location of the motor hotspot based on the brainwave data of the time domains collected for the channels allocated to the plurality of brainwave measuring electrodes 120 or the feature data extracted from the brainwave data.

In the embodiment of FIG. 9, the artificial neural network 136 may include a fully connected layer deep neural network, but the inventive concept is not limited thereto, and it may include various neural networks, such as a convolutional neural network.

The artificial neural network 136 according to the embodiment of FIG. 9 includes input nodes IN, hidden nodes HN, and output nodes ON. The input nodes IN, the hidden nodes HN, and the output nodes ON may be provided as a fully connected layer structure.

Feature data FEAT1, FEAT2, FEAT3, . . . , and FEAT63 for the channels, which have been calculated by the feature data calculating part 134 may be input to the input nodes IN of the artificial neural network 136. In the embodiment of FIG. 9, the feature data calculated for sixty three channels based on the brainwave data collected by sixty three electrodes are input to the input nodes IN of the artificial neural network 136, but the number of the input nodes IN may be variously changed according to the number of the channels (the number of the brainwave measuring electrodes).

A 3-dimensional coordinate of the location of the motor hotspot may be output to the output nodes ON of the artificial neural network 136. The location of the motor hotspot calculated by the artificial neural network 136 may be delivered to the transcranial electrical stimulation electrode 140, and the transcranial electrical stimulation location, to which a current is applied by the transcranial electrical stimulation electrode 140 may be determined according to the location of the motor hotspot.

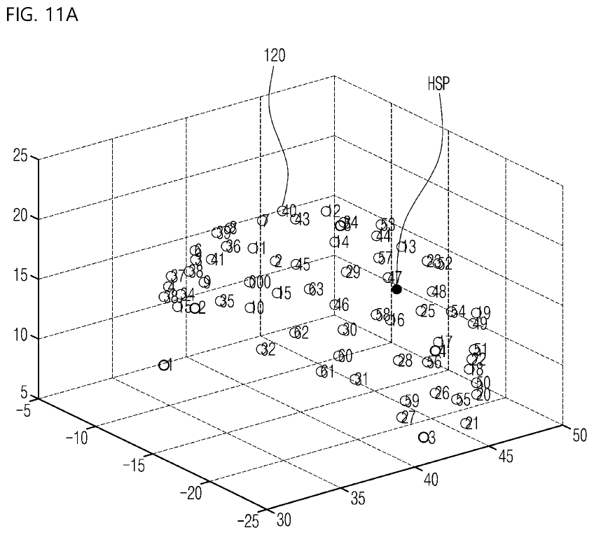

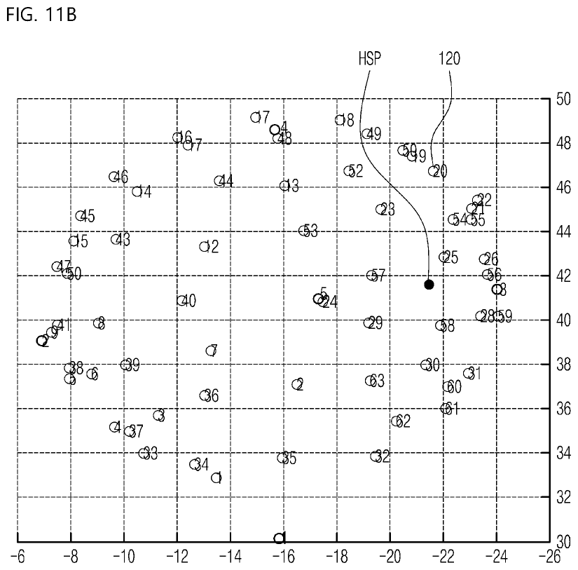

FIG. 10 is a conceptual view illustrating a process of learning the artificial neural network that constitutes the motor hotspot location detecting device using brainwaves according to an embodiment of the inventive concept. FIGS. 11A and 11B are exemplary views illustrating locations of the plurality of brainwave measuring electrodes that constitute the motor hotspot location detecting device using brainwaves according to an embodiment of the inventive concept, and a location of a motor hotspot calculated by a transcranial magnetic stimulation (TMS).

Referring to FIGS. 3, 10, and 11, weight values of the input nodes, the hidden nodes, and the output nodes of the artificial neural network may be learned by inputting the feature data FEAT, such as the power spectrum densities, which have been extracted from the brainwave data collected for the channels by the plurality of brainwave measuring electrodes 120 and the location HSP of the motor hotspot calculated by the transcranial magnetic stimulation TMS into the input nodes of the artificial neural network.

In the example illustrated in FIG. 11, the location HSP of the motor hotspot by the transcranial magnetic stimulation TMS, for example, may be calculated in a scheme of bringing an electrode for measuring the motor evoked potential MEP into contact with a specific body portion, such as a right thumb of the subject person, applying a transcranial magnetic stimulation to a brain portion of the subject person, and finding a transcranial magnetic stimulation application area (a brain/a scalp area portion), in which the motor evoked potential of the specific body portion occurs most explicitly.

Then, in a process of collecting the brainwave data, an exerciser may perform an exercise action, for example, by repeating an exercise subject (an exercise subject of pressing a spacebar once by using a right or left thumb) several times according to a red circle that is displayed on a monitor. In an embodiment, the learning of the artificial neural network may be performed by an artificial neural network learning model (for example, ten-times cross validations) utilizing cross-validations.

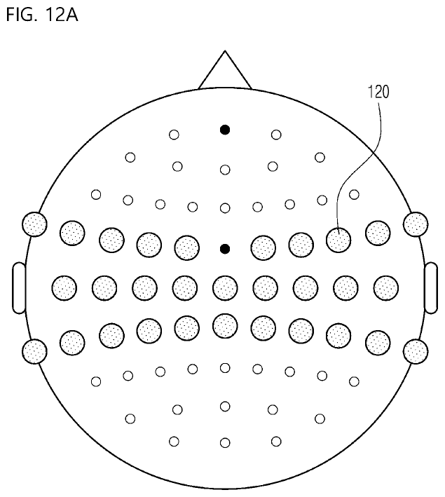

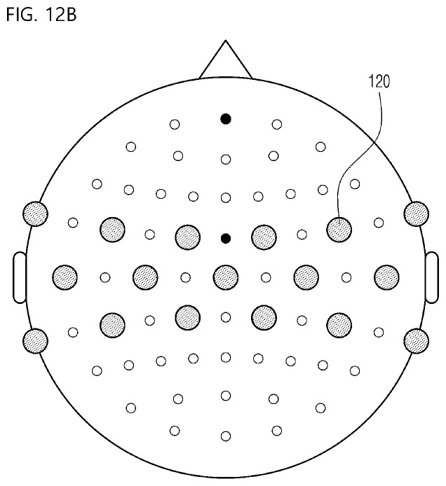

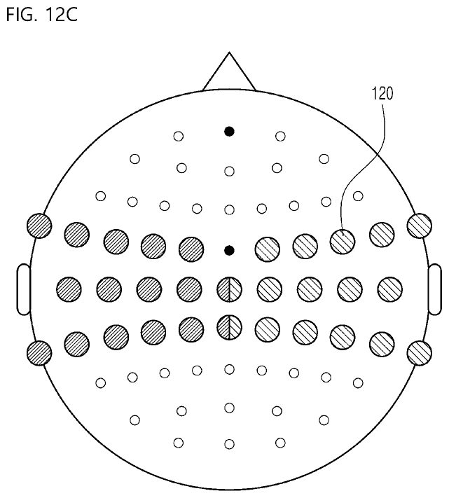

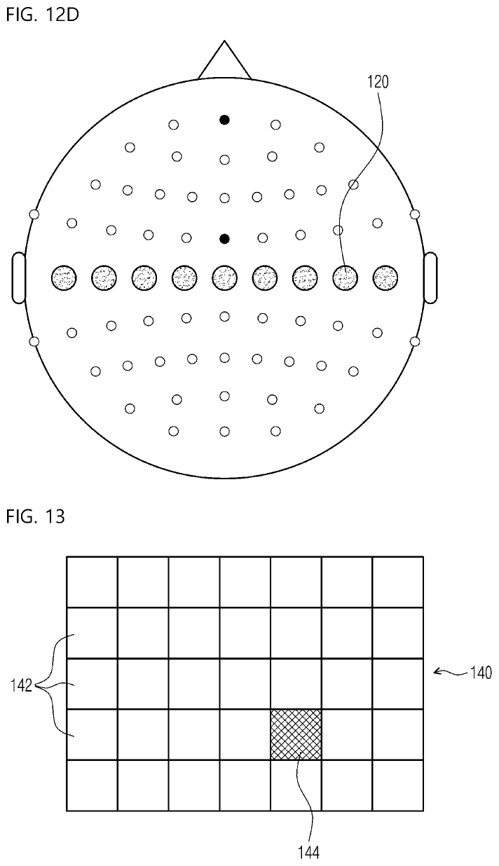

FIG. 12 is an exemplary view illustrating arrangement of a plurality of brainwave measuring electrodes that constitute a motor hotspot location detecting device according to various embodiments of the inventive concept. As illustrated in FIG. 12, when an error is measured through comparison with the location of the motor hotspot in the transcranial magnetic stimulation scheme while the number (the number of channels) of the brainwave measuring electrodes 120 is decreased to less than sixty three channels, it is identified that all the errors is less than 1 cm and correspond to an allowable error range that is smaller than a diameter (a maximum of 1 cm or more) of the electrode for transcranial electrical electrode.

Although the scheme of detecting the location of the motor hotspot based on the power spectrum densities of the brainwave data for the channels has been mainly described in the above embodiment, the location of the motor hotspot may be detected by directly inputting the brainwave data for the channels to the input nodes of the artificial neural network or extracting other feature parameter, except to the power spectrum densities, and inputting them to the input nodes of the artificial neural network.

For example, the feature data calculating part of the motor hotspot location detecting part may calculate the feature data including the correlation coefficient between the brainwave data collected for the plurality of channels and/or the phase synchronization index. The artificial neural network may calculate the location of the motor hotspot based on the feature data including the correlation coefficient and/or the phase synchronization index calculated by the feature data calculating part.

In an embodiment, as illustrated in FIG. 7, the feature data calculating part 134 of the motor hotspot location detecting part may include the correlation coefficient calculating part 1348, and the correlation coefficient calculating part 1348 may calculate the feature data corresponding to the correlation coefficient between the brainwave data collected for the plurality of channels according to Equation 4 as follows.

$$r = \frac{{\sum }_{m} {\sum }_{n} ( {A}_{mn} - \overline{A} ) ( {B}_{mn} - \overline{B} )}{\sqrt{( {\sum }_{m} {\sum }_{n} {( {A}_{mn} - \overline{A} )}^{2} ) ( {\sum }_{m} {\sum }_{n} {( {B}_{mn} - \overline{B} )}^{2} )}} \qquad [ Equation 4 ]$$

In Equation 4, “r” denotes a correlation coefficient between the collected brainwave data, Aₘₙ, and Bₘₙ denote two brainwave data that are correlation coefficient calculation targets, “A” and “B” are averages of the brainwave data, and “m” and “n” (1≤m≤N, 1≤n≤N, N is the number of brainwave data channels, and m and n are integers) denote channel numbers of the brainwave data.

In another embodiment, the feature data calculating part 134 of the motor hotspot location detecting part may include the phase synchronization index calculating part 1350, and the phase synchronization index calculating part 1350 may detect the location of the motor hotspot by calculating the feature data including various network parameters through source localization.

The feature data calculating part 134, for example, may detect the location of the motor hotspot based on the feature data including a phase locking value (PLV) that is a coefficient for determining how much the phases of two signals (brainwave data) are synchronized.

In an embodiment, the feature data calculating part 134 may calculate a phase value by performing a Hilbert transform on the plurality of filtered brainwave signals, and may calculate a phase synchronization index between two phase values based on Equation 5 as follows.

$$\frac{1}{{N}_{fast}} \vert {\sum }_{t = 1}^{{N}_{fast}} {e}^{{Φ}_{1} - {Φ}_{2}} \vert \qquad [ Equation 5 ]$$

In Equation 5, ϕ₁ and ϕ₂ denote phase values of two brainwave signals calculated through the Hilbert transform, N_{fast} denotes a length of measurement time sections of two brainwave signals, and “t” denotes a sample time.

Meanwhile, the brainwave data for the channels, one or two or more of the power spectrum densities (the power spectrum data) obtained by frequency-converting the brainwave data for the channels, the correlation coefficient between the brainwave data, and a source localization based network parameter (a phase synchronization index) may be applied to an input parameter (or input parameters) of the artificial neural network.

FIG. 13 is an exemplary view of the transcranial electrical stimulation electrode that constitutes the transcranial electrical stimulation device according to an embodiment of the inventive concept. Referring to FIGS. 1 and 13, the transcranial electrical stimulation electrode 140 may be provided as a structure, in which a plurality of electrical stimulus application electrodes 142 are arranged on a 2-dimensional or curved surface.

The transcranial electrical stimulation electrode 140 may apply an electrical stimulation to a scalp portion corresponding to the location of the motor hotspot by operating, among the plurality of electrical stimulus application electrodes 142, an electrical stimulus application electrode 144 corresponding to the location of the motor hotspot detected by the motor hotspot location detecting part 130.

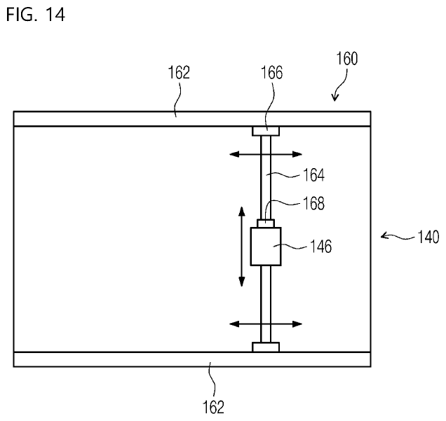

FIG. 14 is an exemplary view of a transcranial electrical stimulation electrode that constitutes a transcranial electrical stimulation device according to another embodiment of the inventive concept. Referring to FIGS. 1 and 14, the transcranial electrical stimulation electrode 140 may include one electrical stimulus application electrode 146.

The transcranial electrical stimulation device may include an electrode moving part 160 that is provided in the head mounted body 110 to move the electrical stimulus application electrode 146 to the location of the motor hotspot.

The electrode moving part 160 may move the electrical stimulus application electrode 146 in a 2-dimensional or 3-dimensional direction on a planar or curved movement surface according to the location of the motor hotspot detected by the motor hotspot location detecting part 130.

In an embodiment, the electrode moving part 160 may include a first driving part 166 that moves the electrical stimulus application electrode 146 in a first direction along a first guide bar 162, a second guide bar 164 that is provided to be movable in the first direction along the first guide bar 162 and is arranged in a second direction that is perpendicular to the first direction, and a second driving part 168 that moves the electrical stimulus application electrode 146 in the second direction along the second guide bar 164.

Furthermore, the electrode moving part 160 may include a third driving part (illustration omitted) that moves the electrical stimulus application electrode 146 in a third direction that is perpendicular to both of the first direction and the second direction. Any driving mechanism may be applied without any particular limitation as long as the driving mechanism that moves the electrical stimulus application electrode 146 may move the electrical stimulus application electrode 146 in the 2-dimensional or 3-dimensional direction.

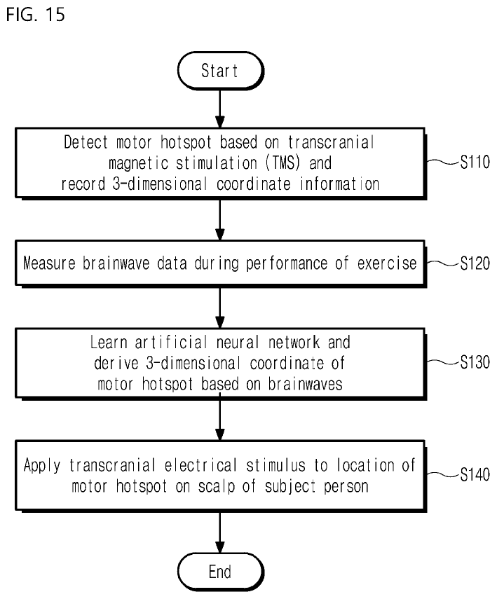

FIG. 15 is a flowchart of a motor hotspot location detecting method using brainwaves according to an embodiment of the inventive concept. Referring to FIGS. 1, 3, and 15, the location (a 3-dimensional coordinate) of the motor hotspot may be measured based on the transcranial magnetic stimulation (TMS) (S110), and the brainwave data according to an exercise action (for example, an action of pressing a spacebar) of a patient may be measured separately to collect the measurement data (S120).

When the measurement data are collected, the artificial neural network model may be learned based on the collected measurement data, and the 3-dimensional coordinate of the motor hotspot may be detected based on brainwaves by using the learned artificial neural network model (S130).

That is, weight values of the input nodes, the hidden nodes, and the output nodes of the artificial neural network 136 may be learned by inputting the feature data, such as the power spectrum densities, which have been extracted from the brainwave data collected for the channels by the plurality of brainwave measuring electrodes 120 and the location of the motor hotspot calculated by the transcranial magnetic stimulation TMS into the input nodes of the artificial neural network 136.

For example, after the artificial neural network 136 performs learning, the brainwave may be collected by measuring the brainwaves generated in the target object at different locations by the plurality of brainwave measuring electrodes 120 provided in the head mounted body 110.

When the brainwave data for the channels are collected by the plurality of brainwave measuring electrodes 120, the location of the motor hotspot may be detected by the artificial neural network 136 based on the brainwave data collected by the plurality of brainwave measuring electrodes 120, by the motor hotspot location detecting part 130.

When the location of the motor hotspot is detected by the motor hotspot location detecting part 130, the transcranial electrical stimulation may be applied to a scalp portion corresponding to the detected location of the motor hotspot by the transcranial electrical stimulation electrode 140 provided in the head mounted body 110 (S140).

The transcranial electrical stimulation electrode 140 may non-invasively apply a current (a transcranial electrical stimulation), such as a fine direct current or alternating current, to a motion hotspot portion that is a brain exercise area of subject person (for example, an exercise rehabilitation patient) whereby an exercise rehabilitation effect of the subject person, such as an exercise function rehabilitation patient, may be enhanced.

Then, an exercise function rehabilitation effect may be maximized by efficiently applying an electrical stimulus to the location of the motor hotspot in a method of selecting any one or more of the plurality of electrical stimulus application electrodes arranged in the head mounted body 110 according to the location of the motor hotspot detected by the motor hotspot location detecting part 130 or moving the transcranial electrical stimulation electrode provided in the head mounted body 110 according to the location of the motor hotspot.

A detailed method for detecting the location of the motor hotspot from the brainwave data collected for the channels by the plurality of brainwave measuring electrodes may be understood through the above description of the embodiment of the motor hotspot location detecting device and the transcranial electrical stimulation device, and thus a repeated description thereof will be omitted.

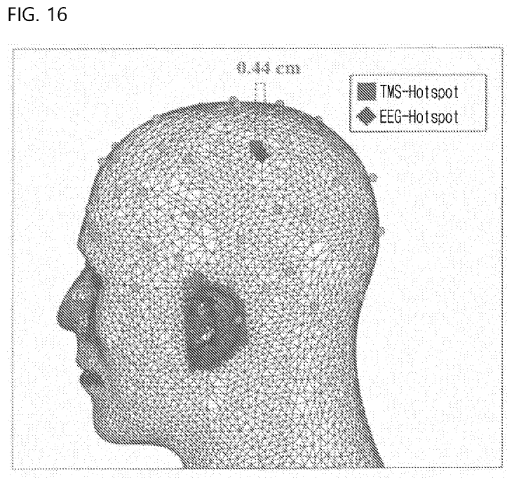

FIG. 16 is a view obtained by comparing a location of the motor hotspot detected through the motor hotspot location detecting method using brainwaves according to an embodiment of the inventive concept and a location of a motor hotspot detected through a transcranial magnetic stimulation (TMS) scheme. As illustrated in FIG. 16, the location (EEG-hotspot) of the motor hotspot detected according to the embodiment of the inventive concept shows a difference of less than 1 cm from the location (TMS-hotspot) of the motor hotspot detected in the transcranial magnetic stimulation scheme, and the difference corresponds to an allowable error range when it is considered that the size (diameter) of the brain electrical stimulation electrode is a minimum of 1 cm or more.

According to the above-described embodiment of the inventive concept, the brainwave data may be collected by the plurality of brainwave measuring electrodes 120 provided in the head mounted body 110 mounted to the subject person by himself or herself, and the precise location of the motor hotspot may be detected.

Furthermore, when the motor hotspot location detecting device and the motor hotspot location detecting method using brainwaves according to the embodiment of the inventive concept are applied to the transcranial electrical stimulation device, the transcranial electrical stimulation may be applied to the detected location of the motor hotspot.

Accordingly, the subject person may obtain an improved exercise rehabilitation effect by applying a brain electrical stimulus to a precise brain exercise area for an individual even though he or she does not visit a hospital to find a brain area, in which a motor evoked potential (MEP) is caused most explicitly due to the transcranial magnetic stimulation (TMS), and inconveniences due to visit to the hospital may be reduced and expenditure of medical costs may be reduced.

In the above-described embodiment, for example, the location of the motor hotspot is determined through a machine learning based algorithm, such as a fully connected layer deep neural network. In this case, to detect the location of the motor hotspot more precisely, big data for learning a neural network model is necessary, and in proportion, a detection speed may be degraded due to an increase in an amount of learning. To prevent the problem, hereinafter, a measure for automatically detecting the motor hotspot of the subject person by a deep learning based algorithm, such as a convolutional neural network, by using only brainwave data for individuals without any database of a high capacity.

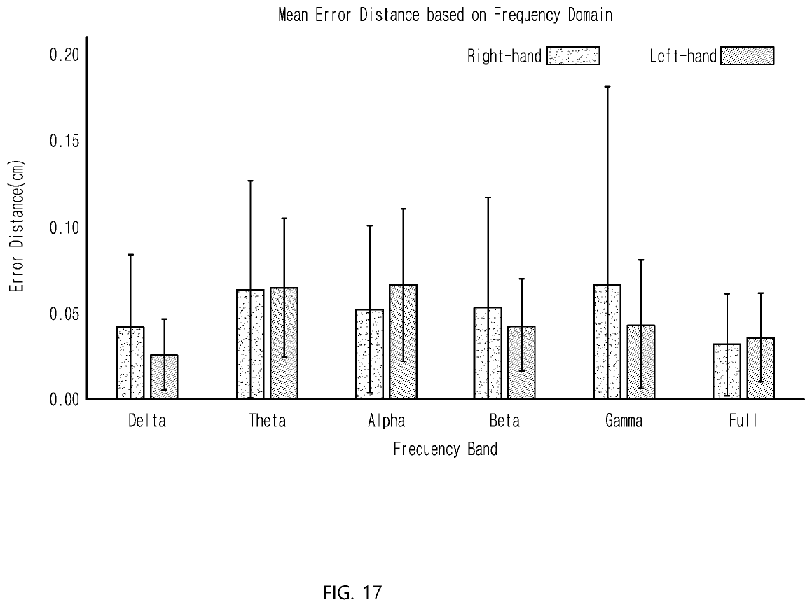

Hereinafter, referring to FIGS. 17 to 20, a transcranial electrical stimulation location determining device and a method based on brainwaves using a convolutional neural network according to another embodiment of the inventive concept will be described. According to the transcranial electrical stimulation location determining device and the method based on brainwaves using the convolutional neural network according to the inventive concept, the target location for the transcranial electrical stimulation for the target object may be automatically determined by using the convolutional neural network based on the brainwave (electroencephalography) (EEG) collected by a user without any database of a high capacity.

According to the embodiment illustrated in FIGS. 17 to 20, because the target location of the transcranial electrical stimulation is determined by using the convolutional neural network based on brainwaves, the target location for the transcranial electrical stimulation may be determined by any person easily and precisely without using big data for generalization of a detection model, and a problem of a detection speed becoming significantly degraded as the amount of learning increases may be solved. Accordingly, the location of the motor hotspot may be detected more promptly and conveniently than in a scheme of constructing a neural network model based on a large amount of data.

FIG. 17 is a graph depicting the transcranial electrical stimulation error calculated based on power spectrum densities after extracting the power spectrum densities corresponding to six frequency bands as features. In the graphs of FIG. 17, delta denotes an error of the location of the transcranial electrical stimulation calculated based on power spectrum densities in a frequency range of 1 to 4 Hz after the power spectrum densities are extracted, theta denotes an error of the location of the transcranial electrical stimulation calculated based on power spectrum densities in a frequency range of 4 to 8 Hz after the power spectrum densities are extracted, and alpha denotes an error of the location of the transcranial electrical stimulation calculated based on power spectrum densities in a frequency range of 8 to 13 Hz after the power spectrum densities are extracted.

Furthermore, beta denotes an error of the location of the transcranial electrical stimulation calculated based on power spectrum densities in a frequency range of 13 to 30 Hz after the power spectrum densities are extracted, gamma denotes an error of the location of the transcranial electrical stimulation calculated based on power spectrum densities in a frequency range of 30 to 50 Hz after the power spectrum densities are extracted, and full denotes an error of the location of the transcranial electrical stimulation calculated based on power spectrum densities in a frequency range of 1 to 50 Hz after the power spectrum densities are extracted.

Then, the errors of the transcranial electrical stimulation location for the frequency bands are obtained through a method of extracting the power spectrum densities for the frequency bands through a process of performing fast Fourier transform (FFT) on the brainwave data collected for the channels by the brainwave measuring electrodes, and comparing the location of the transcranial electrical stimulation obtained by inputting the extracted power spectrum densities for the channels to the convolutional neural network and a location of an exercised brain domain, which is found through the transcranial magnetic stimulation (TMS).

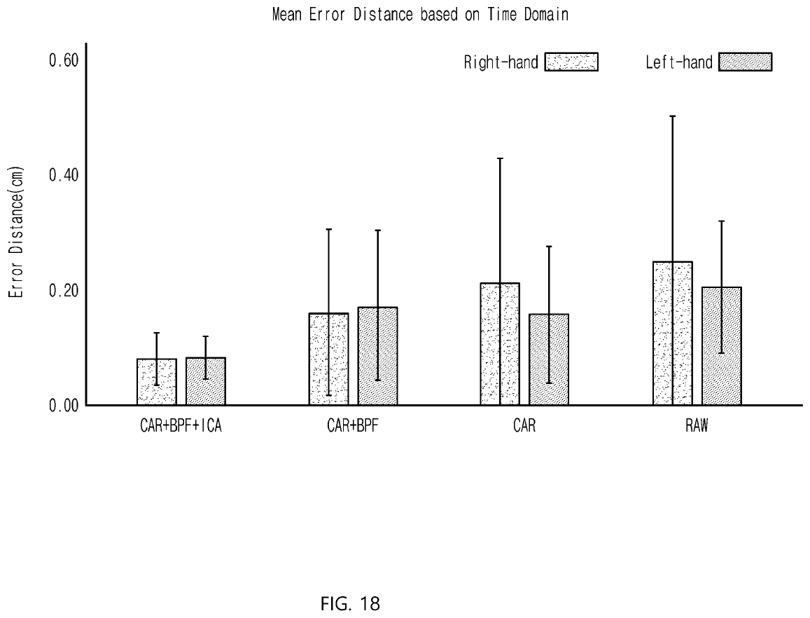

FIG. 18 is a graph depicting an average transcranial electrical stimulation location detection error for pre-treatment operations. It may be identified from the result illustrated in FIGS. 17 and 18 that an average error from the location coordinate of the transcranial electrical stimulation detected by an existing TMS in all conditions regardless of the frequency band is less than 0.1 cm. In particular, an average error of the location coordinate of the transcranial electrical stimulation calculated according to the embodiment of the inventive concept based on all the power spectrum densities including an entire frequency range of 1 to 50 Hz was about 0.03 cm in both cases of a right hand is used and a left hand is used when the TMS is detected. Accordingly, the frequency range of the power spectrum density for extracting the feature data may be freely set to various frequency bands without being limited to the above-mentioned frequency band.

To validate the pre-processing and feature extraction performances of the convolutional neural network together, the pre-processing and the feature extraction are classified into four cases of 1) when an original signal is used (RAW), 2) when noise is removed by using a re-reference scheme (CAR), 3) when noise is removed by using re-reference and band-pass filters (CAR+BPF), and 4) noise is removed by using all of re-reference, a band-pass filter, and an independent component analysis, and a location detection performance (a location error) of the transcranial electrical stimulation was derived in the cases.

As may be seen in FIG. 18, a lower error was shown as the pre-processing operation becomes deep, but a low location error of an average of 0.25 cm or less might be identified even when an original signal that had not been pre-processed is used (RAW). This hints that exercise areas for individuals may be easily detected by anyone without any separate pre-processing process based on determination of an expert when the convolutional neural network is used.

Meanwhile, in the embodiment of FIGS. 17 and 18, the power spectrum densities were extracted as the feature data, but the location of the transcranial electrical stimulation may be detected by extracting the other feature data, such as the correlation between the brainwaves and the phase synchronization index, which have been mentioned above. Furthermore, the power spectrum densities may be extracted through a wavelet transform or an autoregressive scheme, in addition to a fast Fourier transform.

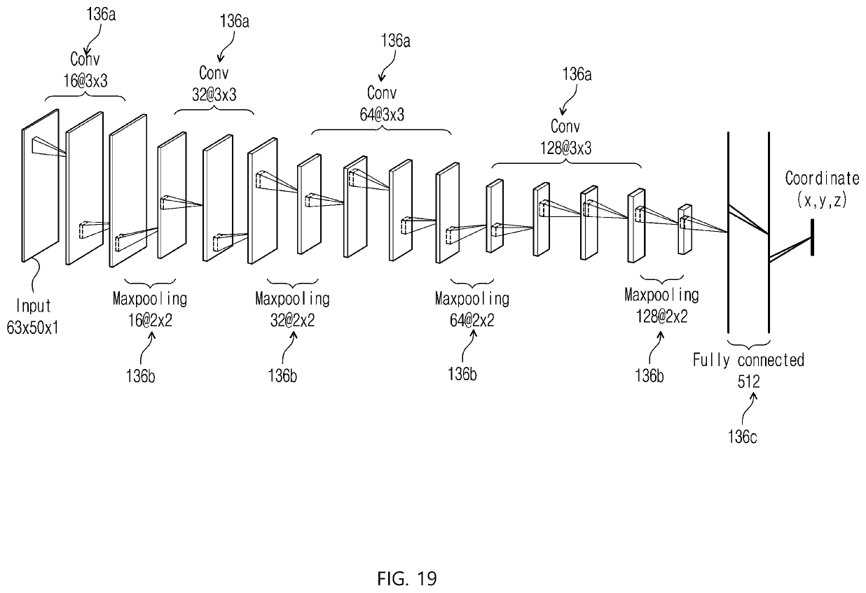

FIG. 19 is a conceptual view of the convolutional neural network that constitutes the brainwave based transcranial electrical stimulation location determining device using a convolutional neural network according to an embodiment of the inventive concept. Referring to FIGS. 4 and 19, the artificial neural network 136 may be implemented by the convolutional neural network. The convolutional neural network may calculate the location of the transcranial electrical stimulation based on the brainwave data of the time domains collected for the channels allocated to the plurality of brainwave measuring electrodes 120 and the feature data extracted from the brainwave data.

The convolutional neural network illustrated in the embodiment of FIG. 19 may include a plurality of convolutional layers 136a, a plurality of pooling layers 136b, and a fully connected layer 136c, but the inventive concept is not limited thereto.

The plurality of convolutional layers 136a may generate convolutional data by performing a temporal axis and/or spatial axis convolution by using a convolutional filter based on the brainwave data or the feature data extracted therefrom.

The plurality of pooling layers 136b may perform pooling such that the features of the input values are learned to be maximally spotlighted to minimize data loss of the brainwave data, of which the dimensional size is significantly smaller than that of the image, but the inventive concept is not limited thereto.

The fully connected layer 136c may perform a function of classification/regression based on the features extracted based on the brainwave data. The fully connected layer 136c may convert the result derived in the preceding operation to a one-dimensional arrangement to derive a 3-dimensional coordinate value as a final output through regression. That is, the 3-dimensional coordinate value of the motor hotspot may be derived through a regression model in a final output layer by converting the feature matrix extracted from the convolution layer of a preceding operation of the fully connected layer 136c to a form of a feature vector.

In the embodiment illustrated in FIG. 19, the feature data of a size of 63 (the number of channels)×50 (the input size of all the frequency bands) from the brainwave data of all the frequency bands corresponding to 1 to 50 Hz are input to the convolutional neural network, but the size of the input data may be changed according to the frequency band.

For example, when the power spectrum densities of a delta band are used, the feature data of a size of 63×4 may be input to the convolutional neural network. Furthermore, the power spectrum densities of a theta band, an alpha band, a beta band, and a gamma band are used, feature data of sizes of 63×5, 63×6, 63×18, and 63×21 may be input, but the inventive concept is not limited thereto.

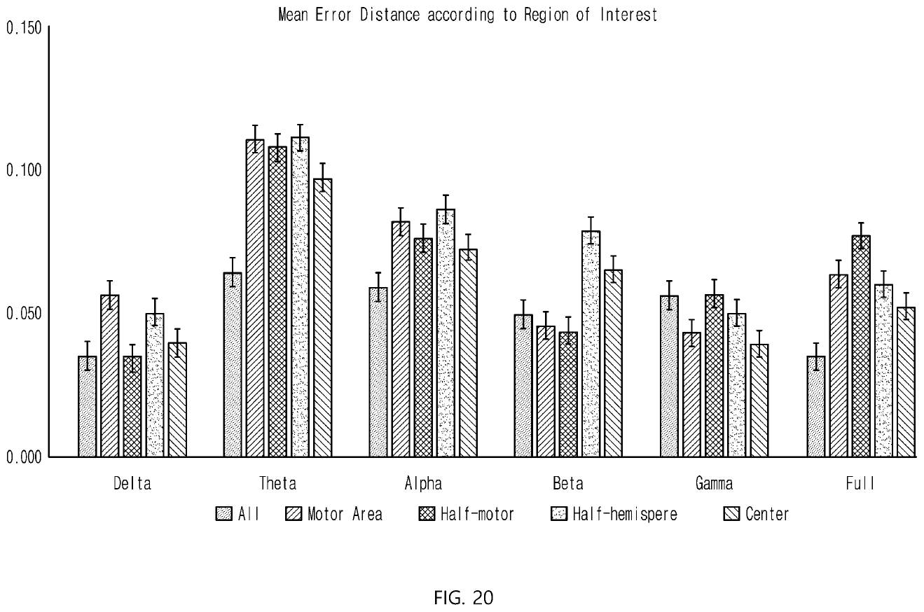

FIG. 20 is a graph depicting a transcranial electrical stimulation location error calculated according to an embodiment of the inventive concept while the number of channels applied to an analysis is gradually decreased from a total of sixty three channels to nine channels with respect to a motion area. Errors of the right hand and the left hand are not statistically different and thus the results for the two hands were averaged. As illustrated in FIG. 20, it may be seen that the location of the transcranial electrical stimulation calculated by using the convolutional neural network according to the embodiment of the inventive concept has an error of as low as about 0.1 cm or less.

When the transcranial electrical stimulation location determining device and the method thereof based on brainwaves using the convolutional neural network according to the embodiment of the inventive concept are applied to the transcranial electrical stimulation device, the transcranial electrical stimulation may be applied to the detected location of the transcranial electrical stimulation.

Accordingly, the subject person may obtain an improved exercise rehabilitation effect by applying a brain electrical stimulus to a precise brain exercise area for an individual even though he or she does not visit a hospital to find a brain area, in which a motor evoked potential is caused most explicitly due to the transcranial magnetic stimulation (TMS), and inconveniences due to visit to the hospital may be reduced and expenditure of medical costs may be reduced.

Furthermore, the motor hotspot of the subject person, which is the target location for the transcranial electrical stimulation may be automatically detected easily and precisely by anyone by using a deep learning based algorithm, such as the convolutional neural network, by using only the brainwave data for individuals without any database of a high capacity, and a problem of a detection speed becoming significantly degraded as the amount of learning increases may be solved.

At least some of the configurations of the above-described embodiments may be implemented by hardware components, software components, and/or a combination of hardware components and software components. For example, the apparatus and the components described in the embodiments, for example, may be realized by using one or more general-purpose computer or a specific-purpose computer such as a processor, a controller, an arithmetic logic unit (ALU), a digital signal processor, a microcomputer, a field programmable array (FPA), a programmable logic unit (PLU), a microprocessor, or any device that may execute and respond to an instruction.

The processing device may perform an operation system and one or more software applications performed on the operating system. Further, the processing device may access, store, manipulate, process, and produce data in response to execution of software. Although one processing device is used for convenience of understanding, it may be easily understood by those skilled in the art that the processing device may include a plurality of processing elements and/or a plurality of types of processing elements.

For example, the processing device may include a plurality of processors or one processor and one controller. Further, another processing configuration, such as a parallel processor, may be possible. The software may include a computer program, a code, an instruction, or a combination of one or more thereof, and the processing device may be configured to be operated as desired or commands may be made to the processing device independently or collectively.

The software and/or data may be embodied in any type of machine, a component, a physical device, virtual equipment, a computer storage medium or device in order to be interpreted by the processing device or to provide an instruction or data to the processing device. The software may be dispersed on a computer system connected to a network, to be stored or executed in a dispersive method. The software and data may be stored in one or more computer readable recording media.

The method according to the embodiment may be implemented in the form of a program instruction that maybe performed through various computer means, and may be recorded in a computer readable medium. The computer readable medium may include a program instruction, a data file, and a data structure alone or in combination thereof. The program instruction recorded in the medium may be designed or configured particularly for the embodiment or may be a usable one known to those skilled in computer software.

Examples of a computer readable recording medium include hardware devices particularly configured to store and perform program instructions, such as magnetic media such as a hard disk, a floppy disk, and a magnetic tape, optical media such as a CD-ROM and a DVD, and memories such as a ROM, a RAM, and a flash memory. Further, an example of the program instruction may include high-level language codes which may be executed by a computer using an interpreter as well as machine languages created by using a compiler. The above-mentioned hardware device may be configured to be operated as one or more software module to perform operations of various embodiments, and the converse is applied.

Although the embodiments of the present disclosure have been described with reference to the limited embodiments and the drawings, the present invention may be variously corrected and modified from the above description by those skilled in the art to which the present invention pertains. For example, the above-described technologies can achieve a suitable result even though they are performed in different sequences from those of the above-mentioned method and/or coupled or combined in different forms from the method in which the constituent components such as the system, the architecture, the device, or the circuit are described, or replaced or substituted by other constituent components or equivalents. Therefore, the other implementations, other embodiments, and the equivalents of the claims pertain to the scope of the claims.

1. A motor hotspot location detecting device using brainwaves for detecting a location of a motor hotspot that is a target location for a transcranial electrical stimulation of a scalp of a target object, the motor hotspot location detecting device comprising:
a plurality of brainwave measuring electrode configured to measure brainwaves generated in the target object at different locations and collect brainwave data; and
a motor hotspot location detecting part configured to detect the location of the motor hotspot based on the brainwave data collected by the plurality of brainwave measuring electrodes.

2. The motor hotspot location detecting device of claim 1, wherein the motor hotspot location detecting part includes:
an artificial neural network configured to calculate the location of the motor hotspot based on brainwave data of time domains, which are collected for channels allocated to the plurality of brainwave measuring electrodes or feature data extracted from the brainwave data.

3. The motor hotspot location detecting device of claim 2, wherein the motor hotspot location detecting part further includes:
a feature data extracting part configured to extract the feature data from the brainwave data collected for the channels, and
the artificial neural network is configured to calculate the location of the motor hotspot based on the feature data extracted from the brainwave data by the feature data extracting part.

4. The motor hotspot location detecting device of claim 3, wherein the feature data extracting part is configured to:
calculate feature data including power spectrum densities for the channels through Furrier transform, wavelet transform, or an autoregressive scheme from the brainwave data of the time domains, which are collected for the plurality of channels allocated to the plurality of brainwave measuring electrodes, and
wherein the artificial neural network is configured to calculate the location of the motor hotspot based on the feature data including the power spectrum densities calculated for the plurality of channels.

5. The motor hotspot location detecting device of claim 3, wherein the feature data extracting part includes:
a correlation coefficient calculating part configured to calculate feature data including a correlation coefficient between the brainwave data collected for the plurality of channels, and
wherein the artificial neural network is configured to calculate the location of the motor hotspot based on the feature data including the correlation coefficient.

6. The motor hotspot location detecting device of claim 3, wherein the feature data extracting part includes:
a phase synchronization index calculating part configured to calculate feature data including a phase synchronization index between the brainwave data collected for the plurality of channels, and
wherein the artificial neural network is configured to calculate the location of the motor hotspot based on the feature data including the phase synchronization index.

7. The motor hotspot location detecting device of claim 2, wherein the artificial neural network performs learning based on the brainwave data collected for the plurality of channels and the location of the motor hotspot measured through a transcranial magnetic stimulation.

8. The motor hotspot location detecting device of claim 2, wherein the artificial neural network includes a convolutional neural network.

9. The motor hotspot location detecting device of claim 1, further comprising:
a head mounted body provided in a form that is mountable on the scalp of the target object,
wherein the plurality of brainwave measuring electrodes are distributed and disposed on an inner surface of the head mounted body such that the brainwave data are collected at different locations on the scalp of the target object.

10. The motor hotspot location detecting device of claim 9, further comprising:
a transcranial electrical stimulation electrode provided in the head mounted body such that the transcranial electrical stimulation is applied onto the scalp of the target object; and
an electrode moving part provided in the head mounted body such that the transcranial electrical stimulation electrode is moved to the location of the motor hotspot.

11. A transcranial electrical stimulation device for detecting a location of a motor hotspot of a target object and applying a transcranial electrical stimulation to a scalp portion corresponding to the location of the motor hotspot, the transcranial electrical stimulation device comprising:
a head mounted body provided in a form that is mountable on the scalp of the target object,
a plurality of brainwave measuring electrodes configured to measure brainwaves generated in the target object at different locations and collect brainwave data and distributed and disposed on an inner surface of the head mounted body;
a motor hotspot location detecting part configured to detect the location of the motor hotspot based on the brainwave data collected by the plurality of brainwave measuring electrodes; and
a transcranial electrical stimulation electrode provided in the head mounted body such that the transcranial electrical stimulation is applied onto the scalp of the target object.

12. The transcranial electrical stimulation device of claim 11, wherein the motor hotspot location detecting part includes:
an artificial neural network configured to calculate the location of the motor hotspot based on brainwave data of time domains, which are collected for channels allocated to the plurality of brainwave measuring electrodes or feature data extracted from the brainwave data.

13. The transcranial electrical stimulation device of claim 11, further comprising:
an electrode moving part configured to move the transcranial electrical stimulation electrode on a planar or curved movement surface in a 2-dimensional or 3-dimensional direction according to the location of the motor hotspot.

14. A motor hotspot location detecting method using brainwaves for detecting a location of a motor hotspot that is a target location for a transcranial electrical stimulation of a scalp of a target object, the motor hotspot location detecting method comprising:
collecting brainwave data obtained by measuring brainwaves generated in the target object at different locations; and
detecting the location of the motor hotspot based on the brainwave data, by a motor hotspot location detecting part.

15. The motor hotspot location detecting method of claim 14, wherein the collecting of the brainwave data includes:
collecting the brainwave data from a plurality of brainwave measuring electrodes, and
wherein the detecting of the location of the motor hotspot includes:
calculating the location of the motor hotspot based on brainwave data of time domains, which are collected for channels allocated to the plurality of brainwave measuring electrodes or feature data extracted from the brainwave data, by an artificial neural network.

16. The motor hotspot location detecting method of claim 15, wherein the detecting of the location of the motor hotspot further includes:
calculating feature data including at least one of power spectrum densities converted from brainwave data in time areas collected for the plurality of channels to frequency area data, and a correlation coefficient and a phase synchronization index between the brainwave data collected for the plurality of channels; and
calculating the location of the motor hotspot based on feature data including at least one of the power spectrum densities, the correlation coefficient, and the phase synchronization index.

17. The motor hotspot location detecting method of claim 15, further comprising:
learning an artificial intelligence model based on brainwave data collected for a plurality of channels and the location of the motor hotspot measured through a transcranial magnetic stimulation and constructing the artificial neural network,
wherein the detecting of the location of the motor hotspot includes:
detecting the location of the motor hotspot by the artificial neural network by using only the brainwave data collected for the plurality of channels, except for the location of the motor hotspot measured through the transcranial magnetic stimulation after the artificial neural network is constructed.

18. The motor hotspot location detecting method of claim 15, wherein the artificial neural network includes a convolutional neural network.

19. The motor hotspot location detecting method of claim 14, further comprising:
moving a transcranial electrical stimulation electrode provided in a head mounted body, in which a plurality of brainwave measuring electrodes are arranged, according to the location of the motor hotspot such that the transcranial electrical stimulation is applied onto a scalp of the target object.

20. A non-transitory computer readable recording medium, in which a program for executing the motor hotspot location measuring method using brainwaves described in claim 14 is recorded.
